(19) United States
(12) Patent Application Publication
(10) Pub. No.: US 2007/0031499 A1
(43) Pub. Date: Feb. 8, 2007

(54) Readily shapeable xerogels having controllably delayed swelling properties

(76) Inventors: Kang Moo Huh, Daejeon (KR); You Mee Choi, Chonbuk (KR); Jae Hyung Park, Gyeonggi (KR); Kinam Park, West Lafayette, IN (US)

Correspondence Address: JAMES H. MEADOWS AND MEDICUS ASSOCIATES, 2804 KENTUCKY, JOPLIN, MO, 64804, US

(21) Appl. No.: 11/495,153

(22) Filed: Jul. 28, 2006

Related U.S. Application Data
(60) Provisional application No. 60/703,126, filed on Jul. 28, 2005.

(51) Int. Cl. A61K 9/14

(52) U.S. Cl. 424/486; 424/487

(57) ABSTRACT

Hydrogels are described which have delayed swelling properties. A hydrogel is formed by reacting a hydrophilic monomer, a first crosslinker, and a second crosslinker. The first crosslinker defines the volume expansion of the hydrogel in an aqueous environment, and the second crosslinker, which is biodegradable, can modulate the swelling rate of the hydrogel in aqueous solution. In its dry state, the hydrogel (xerogel) is flexible and elastic. It can also be cut with a knife or scissors, or molded or shaped by hand. The ready shapeability of the xerogel by trimming or compression affords a superior hydrogel for medical applications.

### REFERENCE TO RELATED APPLICATION

The present application claims the benefit of U.S. Provisional No. 60/703,126, filed Jul. 28, 2005, the disclosure of which is incorporated herein by reference.

### FIELD OF THE INVENTION

The present invention relates to hydrogel compositions, methods of making the same, and their methods of use.

### BACKGROUND OF THE INVENTION

Hydrogels have been used extensively in biomaterials and drug delivery applications. In most cases, useful properties of the hydrogels are based on the swollen form of the hydrogels, i.e., hydrogels that have been exposed to an abundant amount of water. In many cases, however, it is necessary to handle the hydrogels in a dried state before exposing them to aqueous solutions, including body fluids. As used herein, the term “xerogel” refers to a solid formed from a hydrogel by drying.

A recent application of hydrogels has been in the tissue expander area. Tissue expanders have been used to grow extra skin for use in reconstructing various parts of the body. Various forms of tissue expanders have been available since 1957 when the first air-filled rubber balloon was implanted subcutaneously and inflated from outside the body (1). The air was later replaced with a saline solution which was filled into a silicone balloon via a subcutaneously located filling port (2-5). In these models, an increasing volume of air or saline solution had to be introduced to increase the size of the balloon at regular intervals. To make a silicone balloon self-inflatable, the silicone balloon was initially filled with a hypertonic, saturated saline solution, and thus the extracellular tissue fluid permeated through the silicone membrane by osmotic pressure to inflate the balloon (6). In these devices, the silicone membrane has to remain intact to prevent leakage of air, saline solution, or hypertonic, saturated saline solution. Thus, the shape and size of the silicone balloon cannot be altered by cutting, e.g., with scissors or knives.

The osmosis-based self-inflating device became more convenient and useful by using hydrogels made of a copolymer of methyl methacrylate and vinylpyrrolidone (7). Osmed™ Hydrogel Tissue Expanders are commercially available. These osmotically self-inducing expanders hydrate up to 98% in 72 hours (8). This type of device is also called self-filling osmotic expanders (9). These hydrogels in the dry state are glassy and brittle; thus, it is very difficult to change the shape and size of the dried state. Only standard shapes, such as round, rectangular, or crescent shapes, and standard volumes set by the manufacturer, can be used. Clearly, there is a need to develop flexible and elastic tissue expanders made of materials that can be reshaped and adjusted as necessary for each application.

When a xerogel is implanted and exposed to tissue fluid, it starts absorbing aqueous fluid right away. Significant swelling of the xerogel, however, can be delayed for a predetermined time period to provide sufficient time for the wounded area to heal. In theory, the following approaches can be used to provide a delayed swelling property:

1. Xerogel Coated With a Membrane

If a xerogel is coated with a polymer membrane, which limits the absorption of water, the swelling can be delayed accordingly. As the polymer membrane becomes more hydrophobic, the water absorption will be slower. A butadiene-styrene copolymer is an example of a hydrophobic polymer (10). In addition to water-insoluble polymer membranes, lipids can be coated to slow down the water absorption. This particular approach may be useful for microgels. Microgels coated with a lipid bilayer was caused to swell by lipid-solubilizing surfactants or electroporation (11).

2. Xerogel Made of an Interpenetrating Network (IPN) or Semi-IPN

A hydrogel can be synthesized as an IPN or semi-IPN with water-insoluble, but degradable polymers, such as biodegradable poly(D,L-lactic acid) (PLA), poly(D,L-glycolic acid) (PGA), or poly(lactic-co-glycolic acid) (PLGA). For example, a semi-IPN of poly(ethylene glycol) dimethacrylate (PEGDMA) with entrapped PLA forms a hydrogel within the PLA matrix (12). In addition, other biodegradable and elastomeric polymers, such as ε-caprolactone/1,3-trimethylene carbonate copolymer, (13) can be used to inhibit initial swelling of a hydrogel. By controlling the degradation of the PLA or caprolactone matrix, further swelling of the PEG network can be controlled. Such IPN or semi-IPN, however, tends to allow swelling of the PEG network beyond the PLA network, and also it is difficult to change the shape and size of the IPN in the dried state.

3. Xerogel Made of Polyelectrolyte Complexes

A xerogel can be made by electrostatic interactions between a polycation and a polyanion. Non-covalent polyionic complexes can be formed by poly(acrylic acid) (PAA) and chitosan, and the interpolymer complexes can be freeze-dried to produce a xerogel. When this xerogel is placed in an aqueous solution, the presence of higher amount of ions in the medium can result in a network collapse, and thus further swelling (14). In an alternative approach, a polyelectrolyte can be crosslinked with a polyvalent metal ion to form a hydrogel. For example, a polyanion can be reversibly crosslinked with a polyvalent metal cation, and such a cross-link can be dissociated by removing the polyvalent cation using an agent like Na₂HPO₄, di-Na EDTA, and Na hexametaphosphate (15). This type of approach, however, may not provide sufficient osmotic pressure in the body as a gel necessary for use as a tissue expander. Also, they are often too brittle to handle in the dried state.

4. Xerogel With a Degradable Polymer Backbone

Polymers, such as starch, amylase (16) and gelatin (17) can be cross-linked to form hydrogels that can be subsequently dried to form xerogels. As the polymer backbone can be degradable, a xerogel can swell beyond the initial swelling into a hydrogel. However, it is very difficult to control the exact time for delayed swelling as they require enzymes for degradation. Furthermore, degradation of the gel structure will not permit exertion of osmotic pressure to the surrounding tissues.

5. Xerogel With a Degradable Cross-linker

This approach may be most useful as there are numerous biodegradable cross-linking agents available, and their degradation can be controlled. The degradable cross-linker can be prepared by using a variety of methods. First, D and L forms of PLA can be used as a physical cross-linker as the stereocomplex formation can be very strong, and also the formed cross-linker is degradable (18). Other degradable chemical cross-linkers can also be used. They include cross-linkers containing dithiothreitol (19), dithiol (20), or azo bonds that can be degraded by microbial enzymes in the colon (21). These degradable cross-linkers may not be useful when a xerogel has to be implanted into the body. Recently, biodegradable cross-linkers having a polyacid core were used to form a hydrogel with a defined biodegradation rate (22). In addition, oligo-alpha-hydroxy ester cross-linkers were successfully used to control the degradation of the cross-linker, and thus the subsequent swelling of a hydrogel (23). While the use of a biodegradable cross-linker can provide control on the degradation rate, which leads to further, time-dependent swelling, these hydrogels will eventually become water-soluble and thus may not be suitable as tissue expanders. In addition, their xerogels do not have the flexible and elastic properties that are necessary for reshaping and compression in the dry state.

U.S. Pat. No. 4,548,847 (issued to Aberson et al.) proposes a polyelectrolyte hydrogel reversibly crosslinked with a polyvalent metal cation, which reportedly permits delayed swelling characteristics when combined with an agent for removal of the metal cation. U.S. Pat. No. 5,731,365 (issued to Engelhardt et al.) proposes a hydrophilic, highly swellable hydrogel, which is coated with a water-insoluble film-forming polymer. U.S. Pat. No. 6,521,431 (issued to Kiser et al.) proposes a biodegradable crosslinker having a polyacid core covalently connected to reactive groups that can crosslink to polymer filaments.

An object of the present invention is to synthesize xerogels that are flexible and elastic, which can also be mechanically sized and shaped, e.g., with scissors or knives by a clinician, to permit necessary adjustments to each patient. Another object is to provide a controllably delayed swelling property to the xerogel. Since surgery results in damage to the skin and surrounding tissues, it is often necessary to delay swelling of a tissue expander material for several days to a few weeks until the wound area has healed. Thus, an ideal tissue expander material would require the following properties: flexible and elastic properties in the dry state for easy reshaping; ability to be compressed to reduce the size for easy implantation by a short incision into a small pocket with minimal tissue mobilization; no significant swelling for a predetermined time period until the wound area is healed; and a delayed ability to swell and expand the skin.

### SUMMARY OF THE INVENTION

The present invention is directed to a swellable hydrogel that also has elastic, flexible properties when in its dry state, i.e., a xerogel. A hydrogel of the present invention comprises at least one hydrophilic monomer unit that comprises a polymer backbone, a crosslinking agent, and at least one swelling/degradation controller (SDC) moiety. An SDC of the present invention is preferably a polymeric or oligomeric material with a molecular weight less than about 20,000, and it contains at least one chemical linkage cleavable in aqueous solution, which permits the hydrogel to swell at a predefined rate as the SDC degrades by hydrolysis. The SDC can be selected from among polymerizable derivatives of biodegradable moieties, which are incorporated into the hydrogel via radical polymerization. In addition, biodegradable moieties with chemically active functional groups can be chemically incorporated into the hydrogel by condensation reactions. An SDC can be chosen to impart flexible and/or elastic properties to the dried hydrogels (xerogels), also permitting mechanical cutting and shaping.

### DESCRIPTION OF THE DRAWINGS

FIG. 1 shows swelling behaviors of hydrogels based on PEG-DA and PCL-DA at 37° C.:
(a) PEG-DA(Mw=575)/PCL-DA(Mw=1250)=1/1 (w/w), (b) PEG-DA(Mw=700)/PCL-DA(Mw=1250)=1/1 (w/w), (c) PEG-DA(Mw=700)/PCL-DA(Mw=1250)=2/1 (w/w), (d) PEG-DA(Mw=700)/PCL-DA(Mw=1250)=4/1 (w/w).

FIG. 2 shows swelling behaviors of hydrogels based on AA, PEG-DA(Mw=575), and PLA-PEG-PLA-DA (block lengths=750/2000/750) at 37° C. (AA/PEG/SDC=0.4/1/1 by weight).

FIG. 3 shows swelling behaviors of hydrogels based on PEG-DA and PLA-PEG-PLA-DA at 37° C.: (a) PEG-DA(Mw=575)/PLA-PEG-PLA-DA (block lengths=750/2000/750)=1/1 (w/w),
(b) PEG-DA(Mw=575)/PLA-PEG-PLA-DA (block lengths=750/2000/750)=1/2 (w/w), (c) PEG-DA(Mw=575)/PLA-PEG-PLA-DA (block lengths=750/2000/750)=1/4 (w/w).

FIG. 4 shows swelling behaviors of hydrogels based on PLA-PEG-PLA-DA at 37° C.: (a) PLA-PEG-PLA-DA (block lengths=750/2000/750) and (b) PLA-PEG-PLA-DA (block lengths=420/2000/420).

FIG. 5 shows the relative swelling ratios of superporous hydrogels prepared by using salt leaching method and PCL-DA as a SDC: (a) hydrogels based on AA(10 wt %), AAm(15 wt %), Bis(0.25 wt %) and PCL-DA(Mw. 1250, 0.5 wt %) (b) hydrogels based on AA(10 wt %), AAm(15 wt %), Bis(0.25 wt %) and PCL-DA(Mw. 1250, 1.0 wt %) (c) hydrogels based on AA(10 wt %), AAm(15 wt %), Bis(0.25 wt %) and PCL-DA(Mw. 1250, 2.0 wt %).

Abbreviations

AA: Acetic acid
AAc: Acrylic acid
AAm: Acrylamide
AIBN: 2,2′-azobisisobutyrylnitrile
Alginate (Algin): Sodium salt of alginic acid
APS: Ammonium persulfate
BIS: N,N′-methylenebisacrylamide
BPO: Benzoyl peroxide
DW: Distilled water
DMSO: Dimethyl sulfoxide
EBA: N, N′-ethylenebisacrylamide
EG-DA: Ethylene glycol diacrylate
HEA: Hydroxyethyl acrylate
HEMA: Hydroxyethyl methacrylate
MPEG: Monomethoxy poly(ethylene glycol)
NIPAM: N-isopropyl acrylamide
PAA: Poly(acrylic acid)
PAAm: Polyacrylamide
PCL: Poly(ε-caprolactone)
PCL-DA: Poly(ε-caprolactone) diacrylate
PEG-DA: Poly(ethylene glycol) diacrylate
PEG: Poly(ethylene glycol)
PLA: Poly(D,L-lactide), Poly(L-lactide), or Poly(D-lactide)
PLA-DA: PLA diacrylate
PLGA: Poly(lactide-co-glycolide)
PLGA-DA: PLGA diacrylate
PVOH: Poly(vinyl alcohol)
SDC: Swelling/degradation controller
TEMED: N,N,N′,N′-tetramethylethylenediamine

### DESCRIPTION OF THE INVENTION

The present invention entails synthesis of a new class of hydrogels that exhibit flexible and elastic properties in the dried state (xerogels). Such hydrogels are able to be reshaped in the dried state, e.g., by cutting or molding, and exhibit controlled swelling behavior in an aqueous environment. To overcome the limitations of the approaches described hereinabove, hydrogels have been designed and synthesized with degradable cross-linkers along with non-degradable cross-linkers, which permits delayed swelling with retention of hydrogel properties.

Novel hydrogels are prepared using hydrophilic polymers in the presence of chemical crosslinking agents. At least two types of crosslinking agents are incorporated into the hydrogels: (1) a first crosslinker determines the final degree of swelling in an aqueous solution; and (2) a second crosslinker modulates swelling at a predetermined rate. The first crosslinker is not biodegradable and limits the volumetric expansion of hydrogel, which depends on the crosslinking density. The second crosslinker is biodegradable and can be provided as a biodegradable chemical moiety, monomer or oligomer. A biodegradable crosslinker and/or monomer function as a swelling/degradation controller (SDC), which exhibits different degradation rates depending on chemical structure. The degradation rate of SDCs plays a critical role in controlling the delay time before a hydrogel swells, e.g., in excess of 30 days.

Numerous hydrophilic monomers, oligomers, and polymers are available with various crosslinkers to synthesize a hydrogel of the present invention, which exhibits controlled swelling kinetics. Some of the synthetic routes of hydrogels made with different hydrophilic monomers, oligomers, polymers and crosslinkers are described herein. In general, the hydrogels are synthesized using hydrophilic vinyl monomers for the polymer backbone, conventional crosslinking agents, and SDCs. Preferred hydrophilic monomers for this synthesis include, but not limited to, acrylic acid, acrylamide, N-vinyl-2-pyrrolidone, 2-hydroxyethyl methacrylate, N-isopropylacrylamide, and N-(2-hydroxylpropyl)methacryl amide. Exemplary first crosslinkers include N,N′-methylenebisacrylamide (BIS), ethylene glycol dimethacrylate, and poly(ethylene glycol) di(meth)acrylates with different molecular weights in the range of 200-2,000 kDa. Additional examples of suitable monomers and crosslinkers are disclosed in U.S. Pat. Nos. 5,750,585 and 6,271,278 (issued to Park et al.), and U.S. Pat. No. 6,018,033 (issued to Chen et al.), the disclosures of which are incorporated herein by reference.

The chemical structures of some of the non-degradable monomers and crosslinkers that can be employed in the synthesis of a hydrogel of the present invention are shown in Table 1. Different combinations of the monomers and crosslinkers listed in Table 1 permit synthesis of various hydrogels having different chemical structures and, thus, different final degrees of volumetric expansion (swelling).

TABLE 1
Examples of non-degradable monomers and crosslinkers for
synthesis of hydrogels.

| Monomer | Crosslinker |
|---|---|
| Acrylic acid | N,N′-methylenebisacrylamide |
| 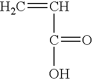 | 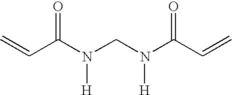 |
| Acrylamide | Ethylene glycol dimethacrylate |
| 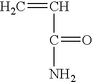 | 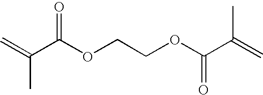 |
| 2-Hydroxyethyl | Poly(ethylene glycol) diacrylates |
| methacrylate | 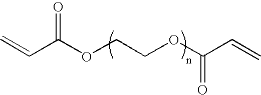 |
| 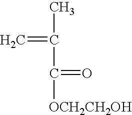 | |
| N-vinyl-2-pyrrolidone | Poly(ethylene glycol) dimethacrylates |
| 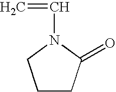 | 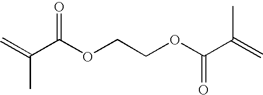 |
| N-(2-hydroxylpropyl) | |
| methacryl amide | |
| 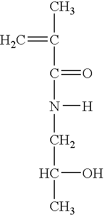 | |

An SDC of the present invention preferably has a polymerizable group at both or one end of the polymer chain and hydrolyzable ester groups in the chain backbone, such as oligomers of poly(lactic acid) (PLA), poly(glycolic acid) (PGA), poly(lactic acid-co-glycolic acid) (PLGA), and poly(ε-caprolactone) (PCL). See Table 2. Such moieties exhibit a degradation behavior that depends upon its hydrophilicity, crystallinity, chemical composition, and molecular weight.

TABLE 2
Representative swelling/degradation controllers (SDCs) for hydrogels.

| Biodegradable crosslinkers and | |
|---|---|
| monomers | Structure |
| Poly(lactic acid)di (meth)acrylate (or mono(meth)acrylate) | 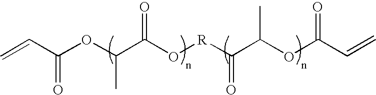 |
| Poly(glycolic acid)di (meth)acrylate (or mono(meth)acrylate) | 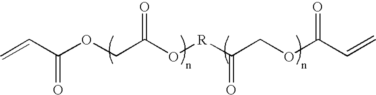 |
| Poly(lactic acid-co-glycolic acid) di(meth)acrylate (or mono(meth)acrylate) | 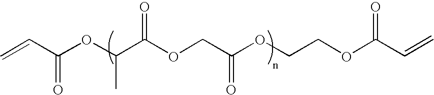 |
| Poly(ε-caprolactone)di (meth)acrylate (or mono(meth)acrylate) | 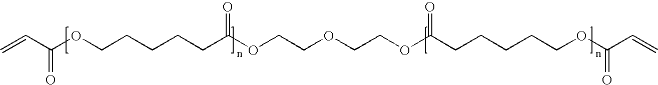 |
| PLA-PEG-PLA di(meth)acrylate (or mono(meth)acrylate) | 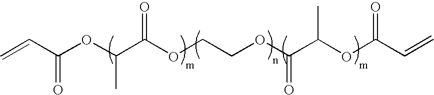 |
| PLA-PEG-PLA di(meth)acrylate (or mono(meth)acrylate) | 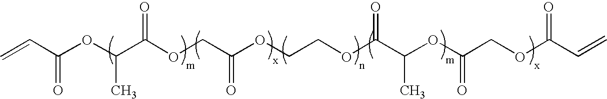 |

Various hydrophilic vinyl monomers can be used to prepare the hydrogel. A series of hydrogels with a broad range of physico-chemical properties can be prepared from many combinational choices of building blocks. A systematic alteration in the chemical composition and structure can lead to better control of physical properties of hydrogels. See Table 3.

TABLE 3
Chemical components for hydrogels showing delayed swelling.

| Components | Chemical name |
|---|---|
| Hydrophilic vinyl | Acrylic acid (AA), Methacrylic acid (MAA), |
| monomers | Acrylamide (AAm), Methacrylamide (MAAm), |
| | Vinylpyrrolidone (VP), Acrylonitrile (AN), |
| | Hydroxyethyl acrylate (HEMA), |
| | Hydroxylpropyl acrylate (HPA), |
| | N-isopropylacylamide (NIPAAm), and other |
| | hydrophililic vinyl monomers. |
| | Vinyl group-containing polysaccharides |
| Crosslinking agents | N,N′-methylenebisacrylamide (BIS), |
| | Poly(ethylene glycol)-di(meth)acrylate, |
| | Polymers having more than two functional |
| | groups for crosslinking reactions. |
| | Vinyl group-containing polysaccharides |
| Initiator and catalyst | AIBN, BPO |
| | APS/TEMED and other redox initiator systems |
| Swelling/Degradation | polymerizable derivatives of biodegradable |
| Controllers (SDCs) | oligomers |

Building Blocks for Synthesis of Hydrogels

Hydrophilic vinyl monomers. The chemical structure and composition of hydrogels can be modified or tailor-made to have desired properties in elasticity, swelling, mechanical strength, degradation, etc. Thus, the choice of hydrophilic vinyl monomers for hydrogels is a primary factor in determining the hydrogel properties. Representative hydrophilic monomers listed in Table 3 can be used as building blocks to construct various kinds of hydrogels with diverse physical properties. However, each monomer may need different conditions for polymerization reaction due to its different reactivity. The hydrogels can also be synthesized using two or more monomers to produce hydrogels composed of copolymers which provide the desired physico-chemical properties.

Cross-linking agents. Not only low molecular weight crosslinking agents but also macromolecules, such as proteins and polysaccharides, can be used as crosslinking agents. Usually three kinds of crosslinking agents are used to make the hydrogels.
a. Bifunctional monomers. N,N′-methylenebisacrylamide (BIS) is a commonly used crosslinking agent for making hydrogels.
b. PEG-(di)acrylates. Poly(ethylene glycol) (PEG) is a well-known hydrophilic polymer, which has been broadly used for biomedical application due to biocompatibility. Bifunctionalized PEGs such as PEG-diacrylate can be used as a crosslinking agent and monofunctionalized PEG such as PEG-acrylate is useful for introducing grafted structure in hydrogels. This type of cross-linker provides flexibility and elasticity to xerogels.
c. Vinyl group-containing polysaccharides. Various kinds of polysaccharides can be modified to have multi-functional vinyl groups that are available for polymerization and crosslinking reaction. For example, water-soluble hydroxyethyl starch (HE-starch) can be modified with glycidyl methacrylate. HE-Starch solution is prepared by dissolving HE-starch powder in PBS solution (10% w/v, pH=8.5). A predetermined amount of glycidyl methacrylate is added to the solution. The heterogeneous mixture solution is kept at 40° C. with stirring for 4 days. The resulting product is precipitated in cold acetone and dried in vacuo overnight.

Swelling/Degradation Controllers (SDCs). SDCs are biodegradable crosslinkers and monomers that can modulate, i.e., regulate in a predetermined way, the swelling rate. The degradation rates of SDCs are dependent on their chemical compositions and structures, and may play an important role in controlling the delay time before hydrogels start swelling.

A diverse class of hydrogels can be synthesized through different combinations of hydrophilic vinyl monomers, crosslinkers, and SDCs. For radical polymerization, benzoyl peroxide or 2,2′-azobisisobutyrylnitrile (AIBN) is preferably used as an initiator. A typical synthetic procedure is shown in Scheme I. In general, to make a hydrogel, hydrophilic vinyl monomer is dissolved in the solvent containing crosslinker, SDCs, and initiator. The mixture is stirred until the solution becomes clear and the reaction is maintained, e.g., at 70° C. for 8 h.

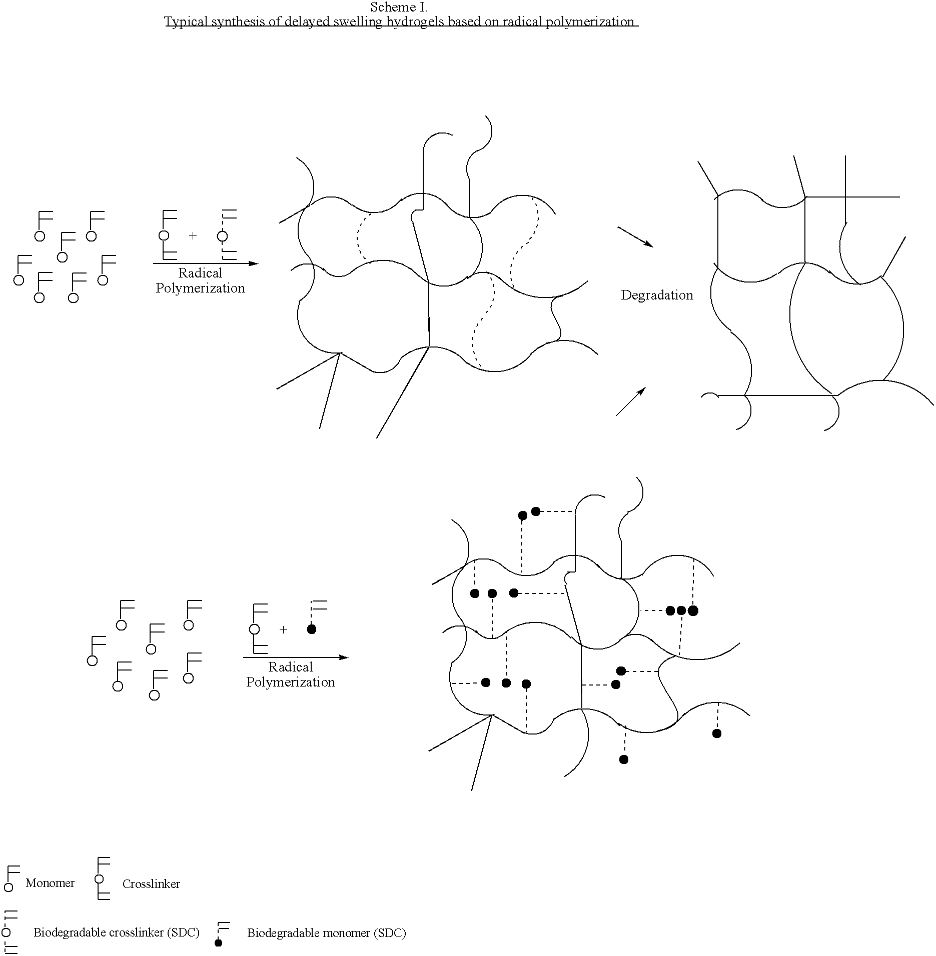

Some particular examples of making flexible, elastic xerogels that use SDCs to firnish a delayed swelling property, are shown below, which illustrate but do not limit the invention.

### EXAMPLES

### Example 1.

Synthesis of PCL-DA

A two-neck flask was purged with dry nitrogen for 20-30 min. PCL diol (5 g) was dissolved in 30 ml of anhydrous benzene and 0.81 ml of acryloyl chloride (or methacryloyl chloride) was dissolved in 20 ml of anhydrous benzene, followed by addition of 1.40 ml of triethylamine. After 20-30 min, the nitrogen purge was stopped and the reaction solution was stirred at 80° C. for 3 h. To remove triethylamine hydrochloride, a side product from the reaction, the reaction solution was filtered. Finally the filtrate was precipitated in an excess of n-hexane and the precipitated product was collected and dried in a vacuum oven for 24 h. The overall reaction is depicted in Scheme 1.

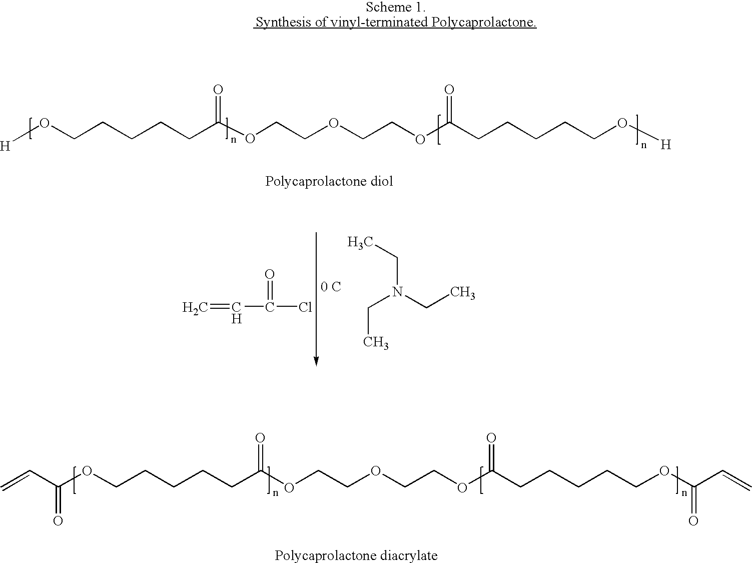

### Example 2

Synthesis of PLGA-DA

A polymerizable PLGA unit was synthesized by introducing a vinyl group at the chain end of PLGA, e.g., by reacting hydroxyl-terminated PLGA with acryloyl chloride, as shown in Scheme 2. One gram of hydroxy-terminated PLGA was dissolved in 10 ml of dichloromethane. Acryloyl chloride (2 equiv. of [OH] in PLGA) was slowly added and the mixture was stirred for 3 h at room temperature. The resulting solution was poured into the excess amount of cold diethyl ether, and the precipitate was filtered, followed by drying under vacuum for 2 days at room temperature.

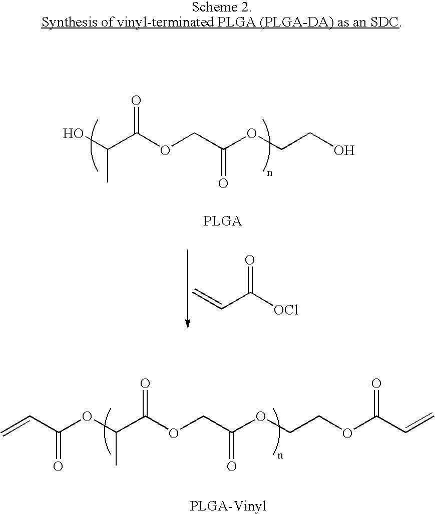

### Example 3

Synthesis of Triblock SDCs for Hydrogel

In addition to the SDCs listed in Table 2, copolymers with two or more different repeating units are useful to precisely control the swelling kinetics and other physical properties of the hydrogel. One example is PEG-PLGA-PEG triblock copolymer. Incorporation of PEG, which has a low glass transition temperature (˜−60° C.), is expected to improve the softness of a xerogel, a dried hydrogel. The overall synthetic scheme for PEG-PLGA-PEG triblock copolymer as an SDC is shown in Scheme 3. One gram of carboxylic acid-terminated PLGA was dissolved in 10 ml of dichloromethane containing 1,3-dicyclohexyl carbodiimide (DCC, 1.2 equiv. of [COOH]) and 4-dimethyl aminopyridine (DMAP, 1.2 equiv. of [COOH]). After adding PEG (2 equiv. of [COOH]), the reaction mixture was stirred for 12 h at room temperature. The precipitated dicyclohexyl urea was filtered off, the solution was poured into cold diethyl ether, and the precipitates were filtered and washed with excess ethyl alcohol. After drying under vacuum at room temperature for 2 days, the PEG-PLGA-PEG terminated with hydroxyl end groups (P—OH) is obtained. P—OH was then dissolved in dichloromethane, to which acryloyl chloride (2 equiv. of [OH] in P—OH) was slowly added. The reaction mixture is stirred for 3 h at room temperature, and poured into cold diethyl ether. The precipitate was filtered, followed by drying under vacuum.

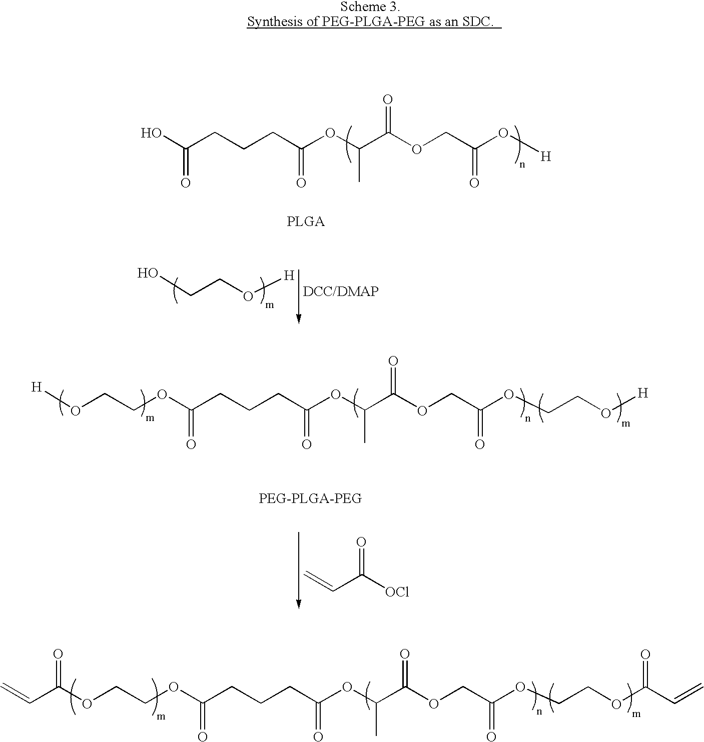

### Example 4

Synthesis of PLA-PEG-PLA as an SDC

PLA-PEG-PLA is another example of an SDC of the invention. A suitable synthetic route is shown in Scheme 4. Prior to the synthesis, PEG was dried for one day at 80° C. under vacuum to remove any moisture. Thereafter, appropriate amounts of PEG and lactide were placed in a one-neck flask. After adding one drop of stannous octoate, the reaction mixture was heated to 150° C. and stirred for 15 h under N₂ atmosphere. The resulting mixture was poured into cold hexane, and the precipitates were filtered and dried for 2 days at room temperature under vacuum to obtain a white powder of PLA-PEG-PLA terminated with hydroxyl end groups (PL-OH). PL-OH was then dissolved in dichloromethane and acryloyl chloride (2 equiv. of [OH] in P—OH) was slowly added. The reaction mixture was stirred for 3 h at room temperature, and poured into cold diethyl ether. The precipitate was filtered, followed by drying under vacuum.

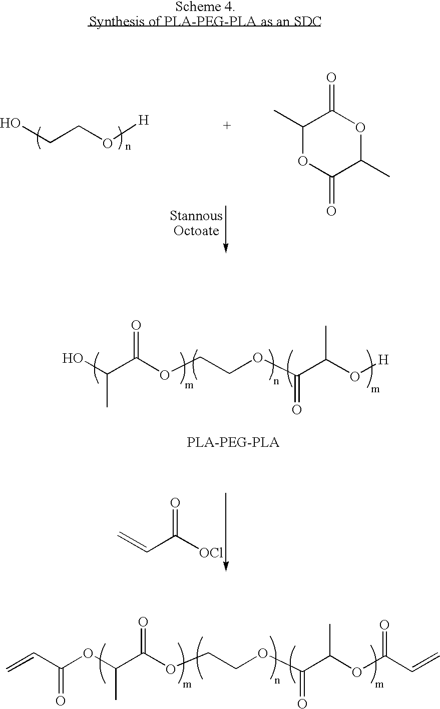

### Example 5

Synthesis of PLGA-PEG-PLGA as an SDC

A suitable synthetic route is shown in Scheme 5. PEG (5 g) was stirred at 150° C. for 3 h under vacuum to remove any moisture. The predetermined amounts of lactide and glycolide were added to the reaction flask and then the mixture was evacuated for 30 min. Subsequently 0.2 ml of stannous octoate diluted with toluene was added and then the reaction mixture was heated up to 155° C. After the reaction for 8 h, the product was poured into cold hexane, and the precipitates were filtered and dried for 2 days at room temperature under vacuum to obtain a white powder of PLGA-PEG-PLGA terminated with hydroxyl end groups (PLGA-OH). PLGA-OH was dissolved in dried dichloromethane containing triethylamine, and acryloyl chloride (2 30 equiv. of [OH]) was slowly added. The reaction solution was stirred at 0° C. for 12 h and then at room temperature for 12 h. The resulting solution was filtered to remove triethylamine hydrochloride and the filtrate was precipitated in cold ether. The precipitate was filtered, followed by drying under vacuum at room temperature for one day.

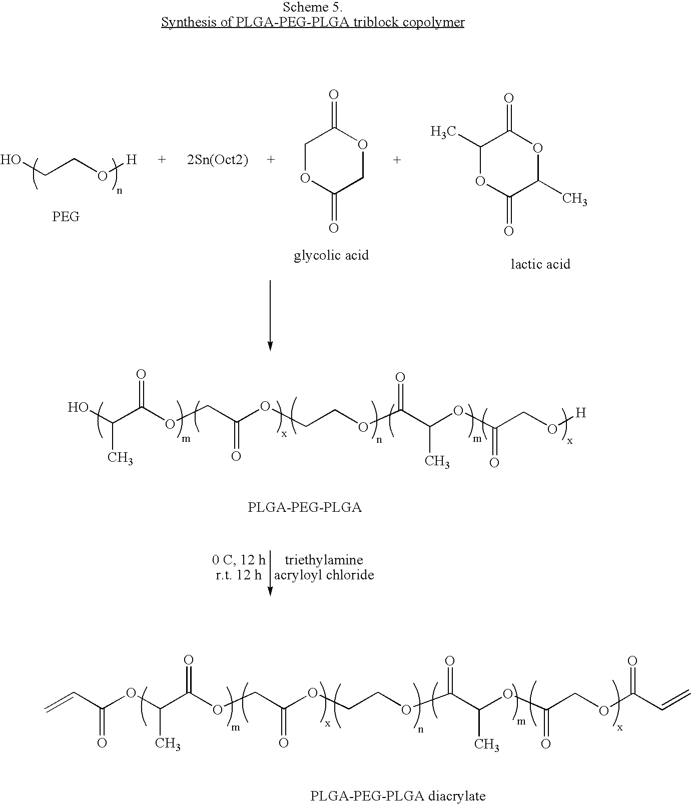

### Example 6

PLA-monoacrylate or PLGA Monoacrylate as an SDC

Some low molecular weight mono-acrylate polymers, such as PLA-monoacrylate and PLGA monoacrylate, can be used as good SDCs. Their hydrophobicity can suppress swelling although they cannot work as a cross-linker. After degradation of hydrophobic moieties, however, hydrogels can start to swell due to enhanced hydrophilicity.

PLA (or PLGA) was dissolved in dried dichloromethane and acryloyl chloride (1.5 equiv. of [OH] in PLA or PLGA) was added to the solution. The reaction solution was stirred 12 h at 0° C. and then 12 h at room temperature. The mixture solution was filtered to remove triethylamine hydrochloride and the filtrate was precipitated in cold ether, filtered, and dried under vacuum for 24 h.

### Example 7

PEG-PLA-monoacrylate or PEG-PLGA Monoacrylate as an SDC

PEG-PLA-monoacrylate is another example of an SDC of the invention. A suitable synthetic route is shown in Scheme 6.

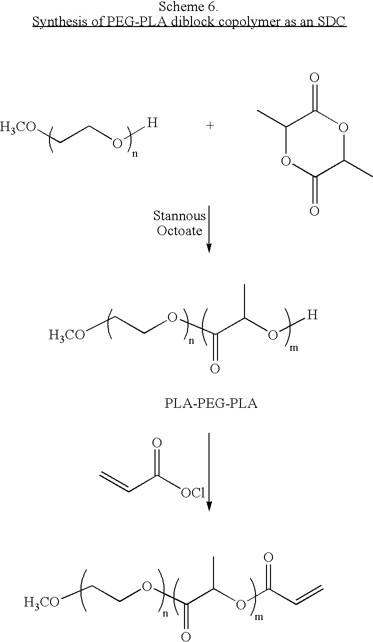

Prior to the synthesis, monomethoxy PEG (MPEG) was dried for one day at 80° C. under vacuum to remove any moisture. Thereafter, appropriate amounts of PEG and lactide were placed in a one-neck flask. After adding one drop of stannous octoate, the reaction mixture was heated to 150° C. and stirred for 15 h under N₂ atmosphere. The resulting mixture was poured into cold hexane, and the precipitates were filtered and dried for 2 days at room temperature under vacuum to obtain a white powder of MPEG-PLA terminated with hydroxyl end groups (MPL-OH). MPL-OH was then dissolved in dichloromethane and acryloyl chloride (2 equiv. of [OH]) was slowly added. The reaction mixture was stirred for 3 h at room temperature, and poured into cold diethyl ether. The precipitate was filtered, followed by drying under vacuum.

### Example 8

Synthesis of Hydrogel Composed of Acrylic Acid, BIS, and PLGA.

In this example, the hydrophilic vinyl monomer, crosslinker, and SDC units are acrylic acid, BIS, and PLGA, respectively. The vinyl-terminated PLGA (PLGA-DA) obtained above and acrylic acid were dissolved in dimethyl sulfoxide. To this solution, the appropriate amounts of BIS as a crosslinker and AIBN as an initiator were added. The mixture solution was heated to 70° C. and allowed to react for 8 h. The hydrogel obtained was washed with excess amounts of diethyl ether and ethyl alcohol, respectively. It was then dried under vacuum at room temperature for 2 days.

The crosslinking density of hydrogel was controlled by the amount of BIS added, whereas the swelling/degradation kinetic was adjusted by varying the amount of PLGA-vinyl and its molecular weight. It should be noted that numerous hydrogels can be prepared in this fashion, in which their characteristics are dependent on the type of monomer, crosslinker, and SDC selected. For example, PCL can be used to prepare hydrogels that show slower swelling than PLGA and PLA. The incorporation of two or more different SDCs can afford two or more onsets of swelling, respectively.

### Example 9

Synthesis of Hydrogels Based on PCL-DA and PEG-DA

In this example, PEG-DA was used for both hydrophilic monomer and crosslinker. PCL is expected to improve the flexibility of dried hydrogel due to its low glass transition temperature (T_{g}) property and also afford biodegradable properties. 0.1 g of diacrylated PCL (Mw. 1250), 0.1 g of PEG-DA (Mw. 575, 700) and 0.007 g of AIBN were dissolved in 2 ml of DMSO and placed into 2 ml microcentrifuge tubes for reaction. The reaction tubes were kept at 65° C. for 12 h. After the reaction, the resultant hydrogels were pulled out gently from the tubes and dried in a vacuum oven for 2-3 days. The MWs and the molar ratios of PEG-DA and PCL-DA can be modulated to control swelling, mechanical, and degradation properties of the hydrogels. Table 4 shows various compositions of hydrogels based on PEG-DA and PCL-DA.

TABLE 4
Various compositions of hydrogel
based on PEG-DA and PCL-DA.

| Sample | PEG-DA | PCL-DA | PEG:PCL(by weight) |
|---|---|---|---|
| 1 | PEG₍₅₇₅₎-DA | PCL₍₁₂₅₀₎-DA | 1:1 |
| 2 | PEG₍₅₇₅₎-DA | PCL₍₁₂₅₀₎-DA | 2:1 |
| 3 | PEG₍₅₇₅₎-DA | PCL₍₁₂₅₀₎-DA | 3:1 |
| 4 | PEG₍₅₇₅₎-DA | PCL₍₁₂₅₀₎-DA | 1:2 |
| 5 | PEG₍₅₇₅₎-DA | PCL₍₁₂₅₀₎-DA | 1:3 |
| 6 | PEG₍₇₀₀₎-DA | PCL₍₁₂₅₀₎-DA | 1:1 |
| 7 | PEG₍₇₀₀₎-DA | PCL₍₁₂₅₀₎-DA | 2:1 |
| 8 | PEG₍₇₀₀₎-DA | PCL₍₁₂₅₀₎-DA | 4:1 |

a) The total monomer concentration was kept at 10 wt %.
b) Hydrogel formation was impossible below the monomer concentration of 5 wt %.

### Example 10

Synthesis of Hydrogels Based on Acrylic Acid(AA), PEG-DA, and PCL-DA

In this example, the hydrophilic vinyl monomer, crosslinker, and SDC units are acrylic acid, PEG-DA, and PCL-DA, respectively. 0.1 g of PCL-DA(Mw. 1250) and 0.1 gram of PEG-DA (Mw. 575) were dissolved in 2 ml of DMSO in 2 ml microcentrifuge tubes. 0.1 gram of AA and 0.007 gram of AIBN were added to the mixture. After sealing with Teflon tape, the mixture tube was placed into a heating oven at 65° C. for 12 h. The resultant hydrogels were dried in a vacuum oven at room temperature for 2-3 days. Various compositions can be applied by varying the MW and the feed ratio of hydrophilic monomer, PEG-DA and PCL-DA to modulate the hydrogel properties. Some examples are listed in Table 5. Various hydrophilic monomers listed in the previous tables can be used instead of acrylic acid.

TABLE 5
Various compositions of hydrogels based
on acrylic acid, PEG-DA, and PCL-DA.

| Sample | PEG₍₅₇₅₎-DA | PCL₍₁₂₅₀₎-DA | hydrophilic monomer | initiator |
|---|---|---|---|---|
| 1 | 5 wt % | 5 wt % | AA 5 wt % | AIBN |
| 2 | 5 wt % | 5 wt % | AA 10 wt % | AIBN |
| 3 | 5 wt % | 5 wt % | AAm 5 wt % | AIBN |
| 4 | 5 wt % | 5 wt % | AAm 10 wt % | AIBN |

### Example 11

Synthesis of Hydrogels Based on PLA-PEG-PLA-DA and PEG-DA

In this example, PEG-DA acts as both hydrophilic monomer and crosslinker, and PLA-PEG-PLA is used as SDC. 0.25 gram of PLA-PEG-PLA diacrylate and 0.25 gram of PEG-DA were dissolved in 5 ml of DMSO and placed into 15 ml of conical centrifuge tube (17 mm×120 mm). 0.0175 gram of AIBN was added to the solution and then the mixture was poured separately to 2 ml microcentrifuge tubes. The tubes were placed in a vacuum oven at 65° C. for 12 h. The hydrogel was taken out and dried in a vacuum oven at room temperature for 2-3 days. The MWs and the molar ratios of PEG-DA and PLA-PEG-PLA-DA can be modulated to control swelling, mechanical, and degradation properties of the hydrogels. Also, other similar types of biodegradable triblock copolymers such as PLGA-PEG-PLGA and PCL-PEG-PCL can be used instead of PLA-PEG-PLA-DA

### Example 12

Synthesis of Hydrogels Based on AA, PEG-DA, and PLA-PEG-PLA-DA

Predetermined amounts of PLA-PEG-PLA-DA (each PLA block length: 747) and 0.25 gram of PEG-DA(Mw. 575) were dissolved in 5 ml DMSO. 0.1 gram of acrylic acid and 0.0175 gram of AIBN were added to the mixture. The mixture was poured separately into 2 ml microcentrifuge tubes. The tubes were placed in vacuum oven at 65° C. for 12 h. The resultant hydrogels were taken out and dried in a vacuum oven at room temperature for 2-3 days. Various compositions can be applied by varying the MW and the feed ratio of hydrophilic monomer, PEG-DA and PCL-DA to modulate the hydrogel properties. Also, various hydrophilic monomers mention previously can be used instead of acrylic acid.

### Example 13

Synthesis of Hydrogels Based on Poly(Vinyl Alchol) (PVA) Glycidyl Methacrylate (GMA), and PLGA.

Hydrophilic monomer units, which can introduce functional groups into the polymer backbone, can be used for hydrogel synthesis in the presence of a crosslinker and a SDC. Scheme 7 shows a synthetic scheme for such a hydrogel, which is composed of PVA, GMA, and PEG-PLGA-PEG as hydrophilic polymer, crosslinker, and SDC, respectively.

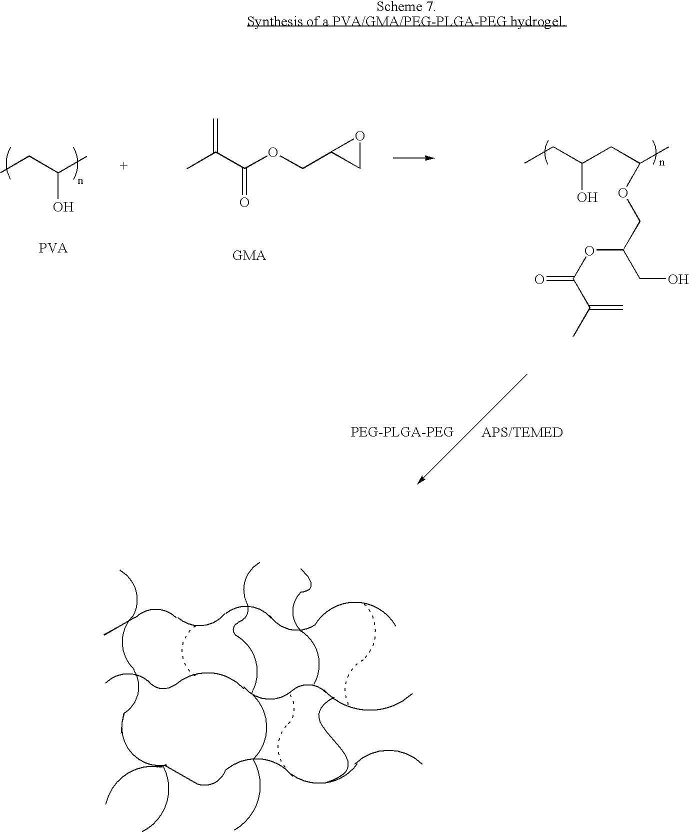

First, PVA and GMA were dissolved in water. After being stirred for 12 h, the solution was dialyzed against excess amount of water for 2 days and freeze-dried for 2 days. PVA-GMA and PEG-PLGA-PEG terminated with vinyl groups, synthesized as described hereinabove, were dissolved in distilled water containing BIS. Ammonium persulfate (APS) and N,N,N′N′-tetramethylethylenediamine (TEMED) were added to initiate the polymerization. The reaction was continued for 1 h, and the hydrogel synthesized was washed with an excess amount of water, followed by drying under vacuum at room temperature for 3 days.

### Example 14

Synthesis of pH-sensitive Hydrogels

Hydrogels may show a pH-sensitive swelling behavior when the SDC contains a linkage cleavable at a certain pH. One example is to introduce a cis-aconityl linkage into the SDC, which is susceptible to hydrolysis at low pH (<˜6.0). Scheme 8 shows a synthetic route for making a PEG-based SDC bearing cis-aconityl acid (SDC-CA). Since there are many hydrophilic polymers possessing hydroxyl and amino groups capable of reacting with carboxylic acid, SDC-CA is useful to be incorporated into a hydrogel intended to exhibit rapid swelling at low pH. A few examples of hydrophilic polymers for this purpose include synthetic polymers, such as PVA, and natural polysaccharides, such as chitosan, alginate, dextran, and hyaluronate.

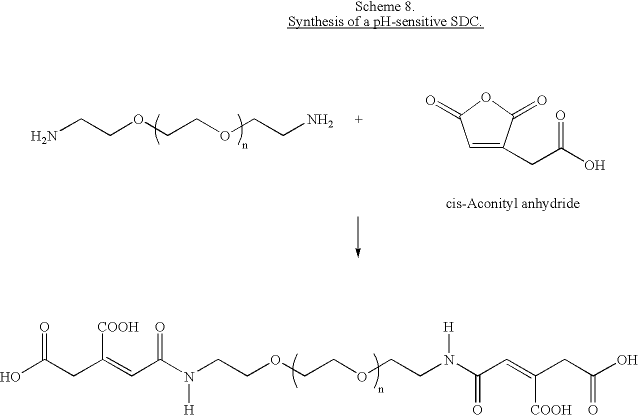

### Example 15

Synthesis of Biodegradable Hydrogels

The backbone of a hydrophilic polymer can be biodegradable. Degradation of hydrogel in biological environments is often very important for biomedical applications, since the hydrogel can be removed without any surgical operation. The biodegradable hydrogel was prepared using biodegradable/hydrophilic polymer (BHP), crosslinker, and SDC. A plurality of BHP products are available for such synthesis, including a synthetic polymer bearing hydrolyzable linkage and natural polysaccharides, such as chitosan, alginate, dextran, and hyaluronate.

One example is to use glycol chitosan as the hydrophilic polymer. Scheme 9 shows a chemical modification of glycol chitosan. Glycol chitosan is dissolved in water/acetone (1:1 v/v) to give a polymer concentration of 1w/v %. Acryloyl chloride is slowly added and the solution is stirred for 3 h. The impurities are removed by dialysis against the excess amount of water for 2 days. Glycol chitosan bearing vinyl group (GC-vinyl) is then obtained after being freeze-dried for 3 days. A number of biodegradable hydrogels can be produced using GC-vinyl by varying the composition of crosslinkers and SDCs, as listed in Tables 1 and 2.

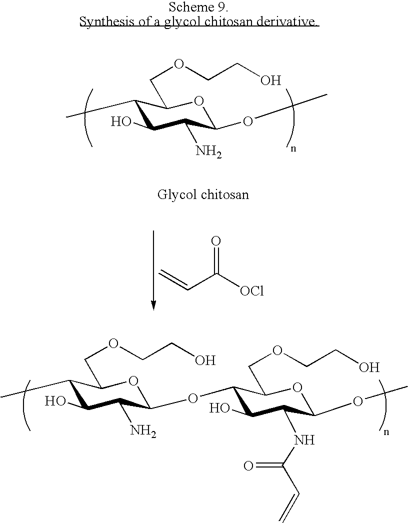

Another example is to use alginate as a hydrophilic backbone, as shown in Scheme 10. Since alginate does not have a primary amino group in the backbone, the chemistry to introduce a vinyl group is different from (glycol) chitosan. In brief, alginate and GMA is dissolved in distilled water, and the solution is stirred for 12 h. The resulting solution is dialyzed against excess amount of water and freeze-dried for 2 days. The alginate-GMA obtained is also useful for syntheses of a plurality of hydrogel systems using different crosslinkers and SDCs.

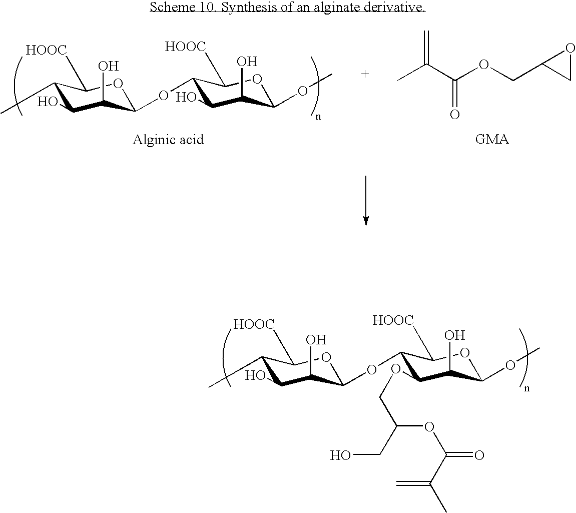

### Example 16

Delayed Swelling Behavior of Hydrogels

To measure the weight swelling ratio, the hydrogels were cut into disk shape (2 mm in diameter and 3 mm in thickness) and then dried in a vacuum oven for 24 hrs to remove any residual moisture. After immersion in an excessive amount of distilled water at room temperature or 37° C. for fixed time periods, the weights of the swollen hydrogels were measured after removal of excess surface water by patting the samples with filter paper. The weight swelling ratio (Sr) of the hydrogels was calculated from the following equation:
Sr=Wₛ/W_{d}
where Wₛ and W_{d} are the weights of the swollen and dried hydrogels, respectively.

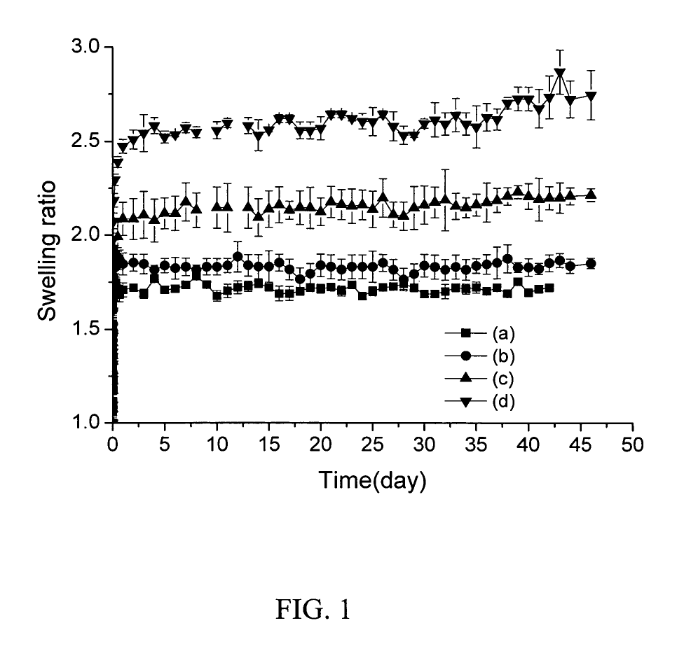

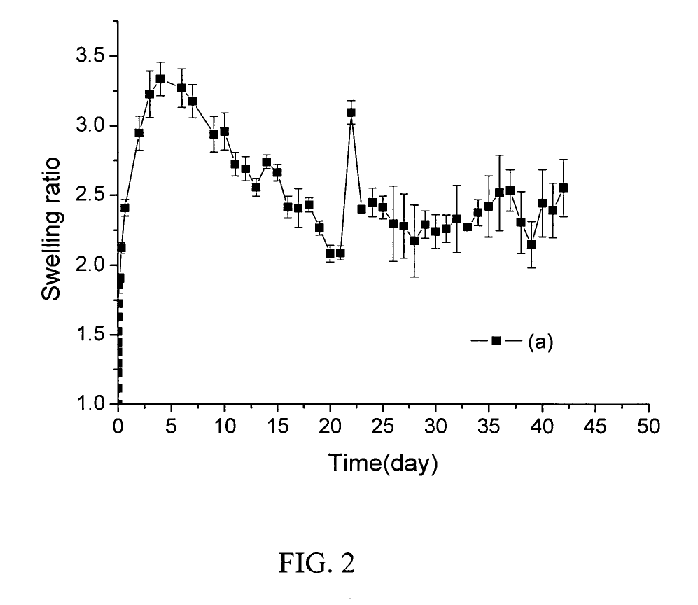

FIGS. 1-4 show the results of swelling tests of several hydrogels. The degradation rates of SDCs were dependent upon the MW and the chemical composition. The degradation rates of SDCs with the same chemical compositions increased with decreasing MW. On the other hand, in case of SDCs with similar MWs the degradation rate increased in the order of PLGA, PGA, PLA, and PCL. So, the delayed time for swelling can be modulated by choosing a SDC with a suitable degradation rate for specific applications.

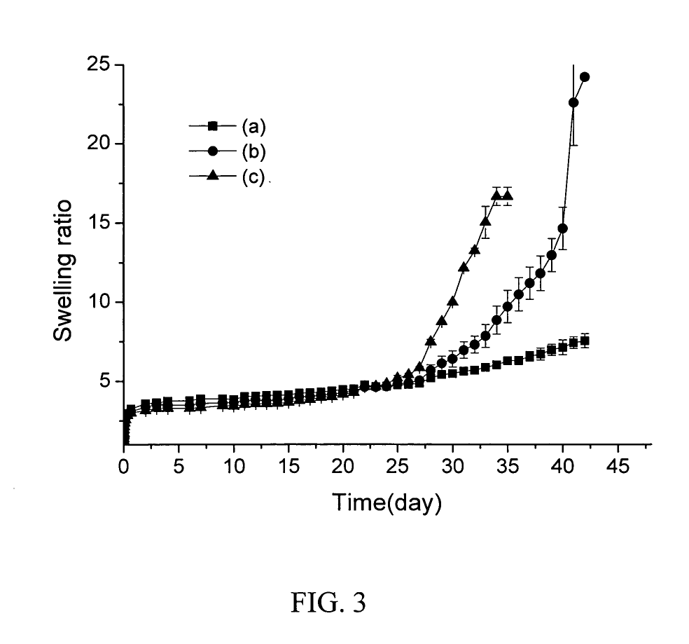

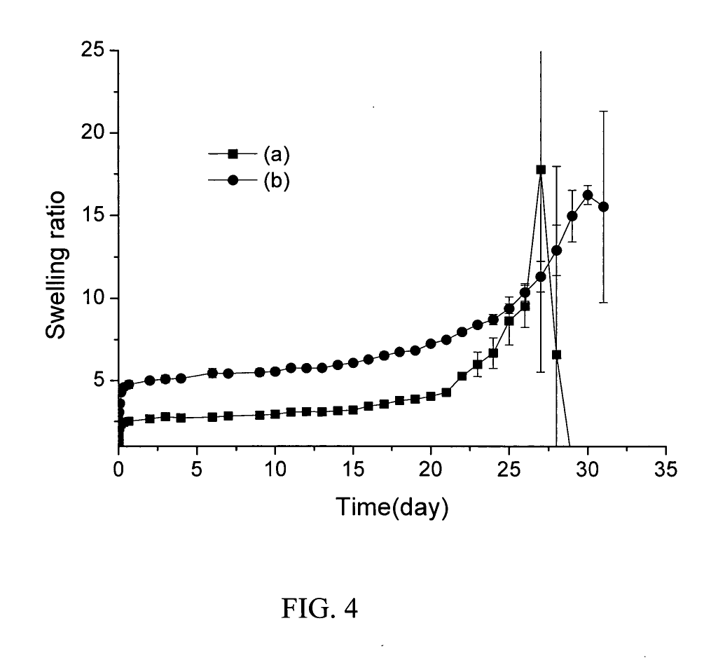

In cases of using PLA-PEG-PLA-DA as a SDC, hydrogels showed the delayed time ranging from 20 to 30 days for swelling (FIGS. 3 and 4). As shown in FIG. 4, the hydrogels made of only PLA-PEG-PLA showed a delayed swelling after 25 days and then dissolved in aqueous media due their complete degradation. Because PCL required a much longer time for degradation, their hydrogels did not show a delayed swelling even after 45 days. Considering the slow degradation nature of PCL, probably more than 2 months is required for delayed swelling.

### Example 17

Superporous Hydrogels Showing Delayed Swelling

Usually, hydrogels take a long time to swell to their equilibrium state. The hydrogels showing delayed swelling behaviors also require several hours to days for their equilibrium swelling. One way to enhance the swelling rate and increase the swelling size is to make them superporous. Because superporous hydrogels (SPHs) can show much faster swelling with higher swelling ratio than other nonporous hydrogels, they can be very useful for demonstrating a hydrogel showing a delayed swelling with fast initial swelling and high osmotic pressure at final swelling stage. Here, two general methods, the gas blowing technique and the salt leaching method, were used for the preparation of superporous hydrogels. When water-soluble SDCs were used, superporous hydrogels were prepared using the gas blowing technique in aqueous media. In cases of using water-insoluble SDCs, the hydrogels were prepared using the salt leaching method in organic phase.

A. Preparation of Superporous Hydrogels Based on AA/AAm Using Gas Blowing Technique

The SPHs were prepared by polymerization of water-soluble monomers, AA and AAm, in the presence of BIS (0.25% w/v) as a cross-linking agent. AA (10% w/v), AAm(15% w/v), BIS (0.25% w/v), and PF127 (0.5% w/v) were dissolved in distilled water. The predetermined amount of a biodegradable SDC was added to the monomer solution. The pH value of the solution was adjusted to 4.5 by adding 8 M NaOH solution. The monomer solutions (8 ml) were placed into polypropylene conical tubes (50 ml) and then APS (0.6% w/v) and TEMED (0.4% w/v) were added. After 3.5 min, sodium bicarbonate powder (5% w/v) was added to the solutions with vigorous stirring using a spatula to generate and distribute gas bubbles evenly throughout the reaction solution. The solutions were kept for 30 min to ensure complete polymerization. The resultant SPHs were dehydrated in ethyl alcohol and placed in a drying oven at 60° C. for 12 h.

B. Preparation of Superporous Hydrogels Based on AA/AAm Using Salt Leaching Method

The SPHs were prepared by polymerization of water-soluble monomers, AA and AAm, in the presence of BIS (0.25% w/v) as a cross-linking agent. AA (10% w/v) and AAm (15% w/v), and BIS (0.25% w/v) were dissolved in DMSO. The predetermined amounts of a biodegradable SDC (PCL-DA, PEG-PLA-PEG-DA, or PLGA-DA) and AIBN were added to the monomer solution. The monomer solution (8 ml) was poured into a polypropylene conical tube (50 ml) containing sodium chloride salt particulates (several hundred micrometers). The reaction solution was placed into in a heating oven at 60° C. for 12 h. The resultant hydrogel was removed from the tube and placed in distilled water to dissolve the salt out. Finally, the hydrogel was dried in a drying oven.

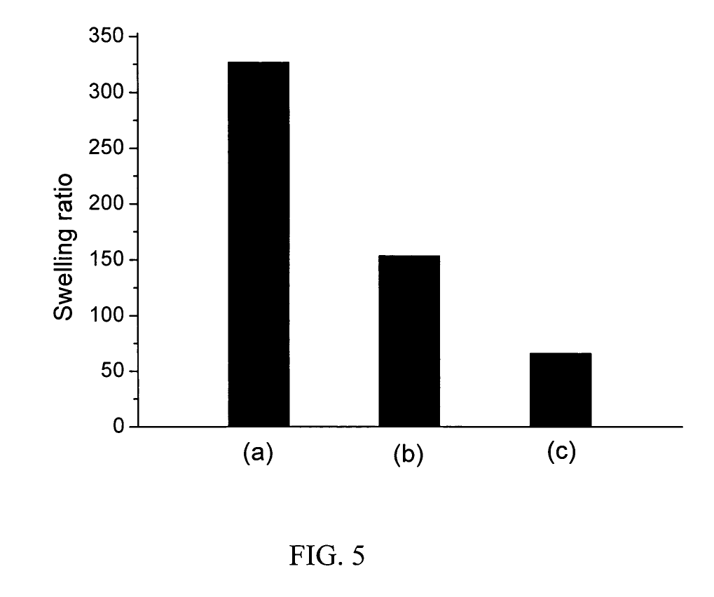

FIG. 5 shows the relative swelling ratios of hydrogels based on AA, AAm, BIS, and PCL-DA. The hydrogels show a lower swelling ratio as the amount of PCL-DA used as a SDC increase. But their swelling ratios are much higher than other typical hydrogels and ranged from several tens to hundreds. So, making the hydrogels superporous can be a good method to enhance the swelling ratio and pressure.

### Example 18

Preparation of SPHs Based on PEG/PCL

PEG-DA (5% w/v) and PCL-DA (5% w/v) were dissolved in DMSO. AIBN were added to the solution and placed into 50 ml of polypropylene conical tube containing sodium chloride salt particulates (several hundred micrometers). The reaction solution was placed in a heating oven at 60° C. for 12 h. The resultant hydrogel was taken out of the tube and placed in distilled water to dissolve the salt out, following by drying in a vacuum oven for 2-3 days. The MWs and the molar ratios of PEG-DA and PCL-DA blocks can be modulated to control swelling, mechanical, and degradation properties of the hydrogels.

### Example 19

Mechanical Properties of Hydrogels

Typical hydrogels, such as those based on AAc, AAm, HEMA, etc., are glassy and brittle in the dry state and thus it is very difficult to change the shape and size of the dried state. Even though the hydrogels can show elastic behavior to some degree in swollen state, their mechanical strength in the swollen state becomes too weak to change their shape by using physical forces or devices such as scissors, knives or scalpels. Therefore, it is very useful to make flexible and elastic hydrogels even in the dried state so that they can be reshaped and adjusted as necessary for each application. PEG is a hydrophilic polymer and its glass transition temperature is very low due to the flexible chain structure. When PEG was used as a building block for preparing hydrogels with other biodegradable polyesters such as PGA, PLA and PCL, the hydrogels can show flexible and/or elastic properties even in the dried state. For instance, a hydrogel, and its xerogel, made of PEG and PCL was flexible and elastic, and remained intact even after application of repeated bending or stretching. The xerogel can be stretched to almost twice the original length without breaking. (Elongation>80%)

The present invention has been described hereinabove with reference to particular examples for purposes of clarity and understanding rather than by way of limitation. It should be appreciated that certain improvements and modifications can be practiced within the scope of the appended claims.

References

The pertinent portions of the following references are incorporated herein by reference.
1. Neumann, C. G. The expansion of an area of skin by progressive distension of a subcutaneous balloon. Plast. Reconstr. Surg. 19: 124-130, 1957.
2. Radovan, C. Development of adjacent flaps using a temporary expander. Plast. Surg. Forum 2: 62, 1979.
3. Argenta, L. Reconstruction of the breast by tissue-expansion. Clin. Plast. Surg. 11: 257-264, 1984.
4. Iversen, A., et al., U.S. Pat. No. 4,685,477, 1987.
5. Foglia, R., Kane, A., Becker, D., Asz-Sigall, J., and Mychaliska, G. Management of giant omphalocele with rapid creation of abdominal domain. Journal of pediatric surgery 41: 704-709; discussion 704-709, 2006.
6. Austad, E. D. and Rose, G. L. A self-inflating tissue expander. Plast. Reconstr. Surg. 70: 588-593, 1982.
7. Wiese, K. G., Heinemann, D. E., Ostermeier, D., and Peters, J. H. Biomaterial properties and biocompatibility in cell culture of a novel self-inflating hydrogel tissue expander. J. Biomed. Mater. Res. Part A 54: 179-188, 2001.
8. Bacskulin, A., Vogel, M., Wiese, K. G., Gundlach, K., Hingst, V., and Guthoff, R. New osmotically active hydrogel expander for enlargement of the contracted anophthalmic socket. Graefe's Archive for Clinical and Experimental Ophthalmology (Albrecht von Graefes Archiv fur klinische und experimentelle Ophthalmologie) 238: 24-27, 2000.
9. Ronert Marc, A., Hofheinz, H., Manassa, E., Asgarouladi, H., and Olbrisch Rolf, R. The beginning of a new era in tissue expansion: self-filling osmotic tissue expander—four-year clinical experience. Plastic and Reconstructive Surgery 114: 1025-1031, 2004.
10. F. Engelhardt, et al., U.S. Pat. No. 5,731,365.
11. Kiser, P. F., Wilson, G., and Needham, D. Lipid-coated microgels for the triggered release of doxorubicin. J. Control. Release 68: 9-22, 2000.
12. Brown Chad, D., Stayton Patrick, S., and Hoffman Allan, S. Semi-interpenetrating network of poly(ethylene glycol) and poly(D,L-lactide) for the controlled delivery of protein drugs. Journal of Biomaterials Science. Polymer Edition 16: 189-201, 2005.
13. Grijpma, D., et al., Method for providing shaped biodegradable and elastomeric structures of 1,3-trimethylene carbonate polymers. WO Patent 2003-EP12425 (2004041318), 2004.
14. De La Torre Paloma, M., Torrado, S., and Torrado, S. Interpolymer complexes of poly(acrylic acid) and chitosan: influence of the ionic hydrogel-forming medium. Biomaterials 24: 1459-1468, 2003.
15. Aberson, G., et al., U.S. Pat. No. 4,548,847, 1985.
16. Dumoulin, Y., Cartilier, L. H., and Mateescu, M. A. Cross-linked amylose tablets containing alpha-amylase: an enzymatically-controlled drug release system. J Control. Release 60: 161-167, 1999.
17. Fukunaka, Y., Iwanaga, K., Morimoto, K., Kakemi, M., and Tabata, Y. Controlled release of plasmid DNA from cationized gelatin hydrogels based on hydrogel degradation. J. Control. Release 80: 333-343, 2002.
18. De Jong, S. J., Van Eerdenbrugh, B., Van Nostrum, C. F., Kettenes-Van Den Bosch, J. J., and Hennink, W. E. Physically crosslinked dextran hydrogels by stereocomplex formation of lactic acid oligomers: degradation and protein release behavior. J Control. Release 71: 261-275, 2001.
19. Dubose John, W., Cutshall, C., and Metters Andrew, T. Controlled release of tethered molecules via engineered hydrogel degradation: model development and validation. Journal of Biomedical Materials Research. Part A 74: 104-116, 2005.
20. Elbert, D. L., Pratt, A. B., Lutolf, M. P., Halstenberg, S., and Hubbell, J. A. Protein delivery from materials formed by self-selective conjugate addition reactions. J. Control. Release 76: 11-25, 2001.
21. Ghandehari, H., Kopeckova, P., and Kopecek, J. In vitro degradation of pH-sensitive hydrogels containing aromatic azo bonds. Biomaterials 18: 861-872, 1997.
22. Kiser, P., et al. U.S. Pat. No. 6,521,431, 2003.
23. Eichenbaum, K. D., Thomas, A. A., Eichenbaum, G. M., Gibney, B. R., Needham, D., and Kiser, P. F. Oligo-alpha-hydroxy ester cross-linkers: Impact of cross-linker structure on biodegradable hydrogel networks. Macromolecules 38: 10757-10762, 2005.

What is claimed is:

1. A hydrogel comprising a hydrophilic polymer backbone, a first crosslinker, and a second biodegradable crosslinker, wherein the first crosslinker determines a final degree of swelling of the hydrogel in an aqueous solution, and the second crosslinker modulates a rate of swelling of the hydrogel in aqueous solution.

2. The hydrogel of claim 1, wherein the hydrophilic polymer is comprised of hydrophilic monomer units polymerized by free radical polymerization.

3. The hydrogel of claim 1, wherein the hydrophilic polymer is comprised of hydrophilic monomer units selected from the group consisting of acrylic acid, acrylamide, 2-hydroxyethyl methacrylate, N-vinyl-2-pyrrolidone, and N-(2-hydroxylpropyl)methacryl amide.

4. The hydrogel of claim 1, wherein the first crosslinker is a hydrophilic divinyl compound.

5. The hydrogel of claim 1, wherein the first crosslinker is selected from the group consisting of N,N′-methylenebisacrylamide (BIS), ethylene glycol dimethacrylate, and poly(ethylene glycol) di(meth)acrylates having a molecular weight in the range of 200-2000 kDa.

6. The hydrogel of claim 1, wherein the second crosslinker is a biodegradable oligomer or polymer.

7. The hydrogel of claim 6, wherein the biodegradable oligomer or polymer has a molecular weight less than about 20,000 kDa and which contains a linkage cleavable in aqueous solution.

8. The hydrogel of claim 1, wherein the second crosslinker contains an oligomer selected from the group consisting of poly(lactic acid) (PLA), poly(glycolic acid) (PGA), poly(lactic acid-co-glycolic acid) (PLGA), poly(ε-caprolactone) (PCL), chitosan, and alginate.

9. The hydrogel of claim 1, which hydrolyzes over at least 30 days at physiological pH with controlled degradation kinetics.

10. The hydrogel of claim 1, which is flexible and elastic in its dried state.

11. The hydrogel of claim 10, which can be cut mechanically.

12. The hydrogel of claim 10, which can be rolled or compressed by hand.

13. A method of making a hydrogel that exhibits delayed swelling and/or degradation in aqueous solution, comprising:
(a) admixing a hydrophilic monomer, a first crosslinker, and a second crosslinker, wherein each molecule contains at least one polymerizable vinyl group, and wherein the second crosslinker is capable of modulating a rate of swelling of the hydrogel in aqueous solution; and
(b) initiating a radical polymerization reaction to produce the hydrogel.

14. The method of claim 13, wherein the hydrophilic monomer is selected from the group consisting of acrylic acid, acrylamide, 2-hydroxyethyl methacrylate, N-vinyl-2-pyrrolidone, and N-(2-hydroxylpropyl)methacryl amide.

15. The method of claim 13, wherein the first crosslinker is selected from the group consisting of N,N′-methylenebisacrylamide (BIS), ethylene glycol dimethacrylate, and poly(ethylene glycol) di(meth)acrylates having a molecular weight in the range of 200-2000 kDa.

16. The method of claim 13, wherein the second crosslinker is a biodegradable oligomer or polymer.

17. The method of claim 16, wherein the biodegradable oligomer or polymer has a molecular weight less than about 20,000 kDA, and which contains a linkage cleavable in aqueous solution.

18. The method of claim 13, wherein the second crosslinker contains an oligomer selected from the group consisting of poly(lactic acid) (PLA), poly(glycolic acid) (PGA), poly(lactic acid-co-glycolic acid) (PLGA), poly(ε-caprolactone) (PCL), chitosan, and alginate.

19. The method of claim 13, further comprising drying the hydrogel.

20. The method of claim 19, wherein the dried hydrogel has elastic, flexible properties and can be mechanically cut.
